(19) United States
(12) Patent Application Publication
(10) Pub. No.: US 2007/0197606 A1
(43) Pub. Date: Aug. 23, 2007

(54) USE OF PPAR AGONISTS AS ANTI-OXIDANTS

(76) Inventors: Frank J. Burczynski, Winnipeg (CA); GuQi Wang, Winnipeg (CA); Yuewen Gong, Winnipeg (CA)

Correspondence Address: ADE & COMPANY INC., 2157 Henderson Highway, WINNIPEG, MB, R2G1P9, US

(21) Appl. No.: 11/677,260

(22) Filed: Feb. 21, 2007

Related U.S. Application Data
(60) Provisional application No. 60/766,961, filed on Feb. 22, 2006.

(51) Int. Cl. A61K 31/4439; A61K 31/426; A61K 31/405; A61K 31/366; A61K 31/22; A61K 31/202; A61K 31/192

(52) U.S. Cl. 514/342; 514/420; 514/460; 514/548; 514/560; 514/369; 514/571

(57) ABSTRACT

As described herein, L-FABP is an antioxidant and as such PPAR agonists such as the fibrate and statin class of pharmacological agents (e.g., clofibrate) can be used to induce FABP which in turn induces antioxidant activity. This anti-oxidant activity may be used to treat or prevent diseases characterized by free radical damage. These include but are by no means limited to cholestasis, cirrhosis, nonalcholic liver disease, fatty liver, drug-induced free radical damage (e.g., anticancer agents, acetaminophen overdose, etc.)

### PRIOR APPLICATION INFORMATION

This application claims the benefit of U.S. Provisional Patent Application 60/766,961, filed Feb. 22, 2006.

### BACKGROUND OF THE INVENTION

Fatty acid binding proteins (FABP) are intracellular lipid-binding proteins that play an important role in long-chain fatty acid trafficking, metabolism, cell proliferation, signal transduction, and protecting cells against the adverse effects of unbound long-chain fatty acids, their CoA and carnitine esters (Glatz and van der Vusse, 1996, Prog Lipid Res 35: 243-282; Storch and Thumser, 2000, Biochimica et Biophysica Acta 1486: 28-44; Zimmerman and Veerkamp, 2002, Cell Mol Life Sci 59: 1096-1116; GuQi Wang et al., 2004, Mol Cell Biochem 262: 41-49). Tissue-specific isoforms of FABP include heart (H-FABP), liver (L-FABP), intestinal (I-FABP), and brain (B-FABP), etc. Although they are similar in protein structure and function, they are encoded by different genes located on different chromosomes (Zimmerman and Veerkamp).

Liver fatty acid binding protein contains seven methionine and one cysteine group in its amino acid sequence (Thompson et al., 1999). It has been postulated that this protein functions as an intracellular buffer of long chain fatty acids and their CoA and carnitine esters thus maintaining a low concentration of their unbound form. Liver fatty acid binding protein also has been suggested to trap or scavenge cytotoxins and superoxide species, thus protecting cells from reactive oxygen species (Ek-Von Mentzer et al., 2001; Kaikaus et al., 1993; Khan and Sorof, 1990; Luebker et al., 2002). Because liver fatty acid binding protein forms a large portion of the intracellular protein pool and contains a large number of methionines and cysteine, it may have an important function as a cytoprotectant (Levine et al., 1999; Thomas et al., 1995). We previously reported that Chang liver cells were devoid of liver fatty acid binding protein. Transfecting those cells with liver fatty acid binding protein cDNA produced a new stably transfected cell line. Inducing oxidative stress in the liver fatty acid binding protein cDNA transfected and vector transfected cells, we reported that the liver fatty acid binding protein cDNA transfected cells were associated with lower reactive oxygen species levels than the same cells transfected with the vector (Wang et al., 2005), suggesting that the protein indeed has important intracellular antioxidative properties.

Recent studies have indicated that several diseases have been associated with malfunction or deficiency of cellular lipid binding proteins, such as hyperlipidemia, diabetes, and atherosclerosis. Liver disease is one of the most common diseases worldwide with cholestatic liver disease being of a major concern in the North American population. The mechanism of cholestatic liver disease is not well understood. Several hypotheses have been proposed including the involvement of oxidative stress (Ljubuncic et al., 2000, Gut 47: 710-716; Aboutwerat et al., 2003, Biochim Biophys Acta 1637: 142-150). According to the oxidative stress hypothesis, endogenous anti-oxidant systems could prevent liver damage during cholestatic liver disease progression. The experimental model used widely to study cholestatic liver disease is the bile duct ligation (BDL) model (Kountouras et al., 1984, Brit J Exp Path 65: 305-311). It has been reported that BDL is associated with decreased antioxidant activities of hepatic catalase, superoxidase (SOD) and glutathione peroxidase (GTPx) (Orellana et al., 2000, Comp Biochem Physiol C Toxicol Pharmacol 126: 105-111). Moreover, liver mitochondria antioxidative capacity and glutathione (GSH) are decreased in bile-duct-ligated rats (Krahenbuhl et al., 1995, Hepatology 22: 607-612). The exogenous antioxidants, vitamin E (lipophilic) and Trolox (hydrophilic), improved lipid peroxidation and oxidation of glutathione in BDL rats, but had no effect on liver injury (Baron and Muriel, 1999, Biochim Biophys Acta 1472: 173-180). However, it would be interesting to examine other endogenous anti-oxidant systems in the liver. Since L-FABP contains the necessary groups to serve as an antioxidant [Hepatology 38(4):395A, 2003.], it may play an important role in the prevention of cholestatic liver disease. As an important protein involving in long chain fatty acid uptake and metabolism, L-FABP expression in cholestatic liver has not been documented. We demonstrate the expression and anti-oxidative function of L-FABP in an animal model of cholestatic liver disease induced by bile duct ligation (BDL).

In this report we examined the role of liver fatty acid binding protein in a cholestatic liver disease model. The mechanism of cholestatic liver disease is not well understood and several hypotheses have been proposed including the involvement of oxidative stress (Aboutwerat et al., 2003; Ljubuncic et al., 2000). According to the oxidative stress hypothesis, endogenous antioxidant systems could prevent liver damage during cholestatic liver disease progression. The experimental model widely used to study cholestatic liver disease is the bile duct ligation model (Kountouras et al., 1984) which is associated with decreased antioxidant activities of hepatic catalase, superoxide dismutase and glutathione peroxidase (Orellana et al., 2000). Moreover, liver mitochondria antioxidative capacity and glutathione are decreased in bile duct ligated rats (Krahenbuhl et al., 1995). While exogenous antioxidants, vitamin E (lipophilic) and Trolox (hydrophilic) improved lipid peroxidation and oxidation of glutathione in bile duct ligated rats, it had no effect on liver injury (Baron and Muriel, 1999). Whether other endogenous antioxidant systems are available within the liver to improve liver function is not clear. Interestingly, liver fatty acid binding protein levels are known to be reduced in steatosis (Hung et al., 2005). Since liver fatty acid binding protein has been thought to function as an effective antioxidant, it may play an important role in the prevention of cholestatic liver disease. In this report we demonstrated the expression and antioxidative function of liver fatty acid binding protein in an animal model of cholestatic liver disease induced by bile duct ligation.

### SUMMARY OF THE INVENTION

According to the invention, there is provided a method of treating an individual having a disease characterized by free radical damage comprising administering to an individual in need of such treatment an effective amount of a PPAR agonist.

### BRIEF DESCRIPTION OF THE DRAWINGS

FIG. 1. Liver sections from sham and bile duct ligated rats. (A) Normal hepatocytes in portal area from the rat of Sham group. (B) Mild bile duct proliferation and mononuclear cell infiltration in portal area from two weeks post bile duct ligation group. Macrovascuolar cytoplasmic alterations of hepatocytes and many lysed cell areas (arrows in FIG. 1) were observed in this group. Bile duct ligated rats were associated with significant proliferation of bile duct epithelial cells, inflammation and altered liver structure (circle arrow in FIG. 1).

FIG. 2. RT-PCR analysis of L-FABP mRNA at different time intervals following bile duct ligation (+) and sham (−). GAPDH was used as loading control. The optical density of each band was obtained using the NIH Image program. The relative expression of L-FABP mRNA versus GAPDH was displayed against time following bile duct ligation. The significant decrease of L-FABP mRNA level occurred after 2 days of bile duct ligation (**P<0.01). The data represent mean±S.E.M., n=4.

FIG. 3. Western blot analysis of L-FABP levels in bile duct ligated (+) and sham (−) rat livers at different time intervals following bile duct ligation. Actin expression was the loading control. The L-FABP relative expression (optical density units) of each band was obtained using the NIH Image program and expressed as L-FABP versus Actin. Data represent mean±S.E.M., n=4. Statistical changes in L-FABP with time started after 2 days of bile duct ligation, **P<0.01.

FIG. 4. Increased thiobarbituric acid-reactive substances production in liver tissues in bile duct ligated rats. Thiobarbituric acid-reactive substances production in liver was determined by measurement of absorbance at 532 nm using HPLC. The degree of lipid peroxidation was expressed as concentration of thiobarbituric acid-reactive substances in terms of malondialdehyde equivalents per gram of liver proteins. The plot shows a significant thiobarbituric acid-reactive substances increase in bile duct ligated rats compared to sham following 14 days of bile duct ligation. Data represent mean±S.E.M., n=4, **P<0.01.

FIG. 5. Clofibrate reverses reduction of L-FABP mRNA and L-FABP level in bile duct ligated rats. Clofibrate was administered to rats for 5 days (50 mg/day/100 g body weight) by gavage after 7 days bile duct ligation (total bile duct ligation time was 12 days). S represents sham rats (total time was 12 days); B represents bile duct ligated rats (total bile duct ligation time was 12 days); B+C represent bile duct ligated rats with clofibrate treatment. Data represent mean±S.E.M., n=5. Statistical significance was documented by Analysis of Variance with *P<0.05 and **P<0.01.

FIG. 6 Effect of L-FABP on free radicals released by the hydrophilic free radical generator AAPH. L-FABP had as much effect on inactivating hydrophilic free radicals as 3.5 mM Vitamin C, while lysozyme and albumin had negligible effect, 96±1% and 97±1%, respectively (mean±SD, n=6).

FIG. 7 Effect of L-FABP on free radicals released by the lipophilic free radical generator AMVN. L-FABP had as much effect on inactivating lipophilic free radicals as 5 μM Vitamin E (mean±SD, n=6).

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

### Definitions

As used herein, “effective amount” refers to the administration of an amount of a given compound that achieves the desired effect. In this case, an effective amount of a PPAR agonist is an amount sufficient to induce FABP, as discussed above. As will be apparent to one skilled in the art, the effective amount for a given patient may vary according to the age, weight, condition and responsiveness of the patient to the treatment.

As used herein, “purified” does not require absolute purity but is instead intended as a relative definition. For example, purification of starting material or natural material to at least one order of magnitude, preferably two or three orders of magnitude is expressly contemplated as falling within the definition of “purified”.

As used herein, the term “isolated” requires that the material be removed from its original environment.

As used herein, the term “treating” in its various grammatical forms refers to preventing, curing, reversing, attenuating, alleviating, minimizing, suppressing or halting the deleterious effects of a disease state, disease progression, disease causitive agent other abnormal condition.

Cholestatic liver disease is caused by binary obstruction that leads to either primary biliary cirrhosis or primary sclerosing cholangitis. Although the mechanism of cholestatic liver disease is not completely understood, oxidative stress has been recognized as an important factor in the disease process. Thus, endogenous antioxidant systems may have a central role in preventing the liver from extensive damage. We recognized that liver fatty acid binding protein (L-FABP) contains several methionine and cysteine groups which have the potential to function as antioxidants. In this study we demonstrated the expression and anti-oxidative function of L-FABP in an animal model of cholestatic liver disease induced by bile duct ligation (BDL).

L-FABP has an important role as an antioxidant function during hepatocellular oxidative stress. L-FABP expression may be important in preventing the progression of cholestatic liver disease. Furthermore, FABP exists in many tissues and as such regulation of the expression of FABP as described herein is a treatment for many diseases, in addition to liver or heart disease. For example, in some forms of drug overdose, the liver dies because of toxicitiy (free radical damage coming from the offending drug). However, FABP protects the liver from the free radical damage meaning that manipulation of FABP levels is a treatment for drug overdoses. For example, clofibrate may be administered to protect the liver from acetaminophen overdose.

As described herein, L-FABP is an antioxidant and as such PPAR agonists such as clofibrate can be used to induce FABP which in turn induces antioxidant activity. This anti-oxidant activity may be used to treat or prevent diseases characterized by free radical damage. These include but are by no means limited to cholestasis, cirrhosis, nonalcholic liver disease, fatty liver, and drug-induced free radical damage (e.g., anticancer agents, acetaminophen overdose, etc.)

As will be appreciated by one skilled in the art, ‘an individual in need of such treatment’ will be an individual having or suspected of having a disease characterized by free radical damage as discussed above. It is of note that such individuals can be identified using means well-known to one skilled in the art, for example, by using liver function tests to determine if one or more tests shows abnormal levels, such as total protein blood levels (normal range −60-80 g/L), albumin levels (normal range 30-50 g/L), alanine transaminase (normal range (15-45 U/L), alkaline phosphatase (normal range 30-120 U/L) and other similar tests. According, in some embodiments, an individual in need of such treatment refers to an individual having or suspected of having at least one of cholestasis, cirrhosis, nonalcholic liver disease, fatty liver, or drug-induced free radical damage, based on physician examination and/or test results such as those discussed above.

Examples of other PPAR agonists are well-known in the art and include, for example, fatty acids such as Linoelic acid, arachidonic acid, (fish oils); hypolipidemic agents such as the fibrate analogues clofbrate, gemfibrozil, fenofibrate, ciprofibrate, bezafibrate, the statin analogues such as lovastatin, pravastatin, simvastatin, mevastatin, fluvastatin, and the like; hypoglycemic agents such as rosiglitazone, troglitazone, the nonsteroidal anti-inflammatory agents such as indomethacin, fenoprofen, and ibuprofen.

As will be appreciated by one of skill in the art, these drugs are classified as lipid-lowering agents. They decrease the ‘bad’ lipids. However, their role in activating FABP to act as an antioxidant was not previously known.

In another aspect of the invention, there is provided a method of treating an individual having a disease characterized by free radical damage comprising administering to an individual in need of such treatment an effective amount of a PPAR agonist.

In another aspect of the invention, there is provided a method of activating FABP in an individual in need of such treatment comprising administering to said individual an effective amount of a PPAR agonist.

In another aspect of the invention, there is provided a method of treating cholestasis, cirrhosis, nonalcholic liver disease, fatty liver, or drug-induced free radical damage in an individual in need of such treatment comprising administering to said individual an effective amount of a PPAR agonist, wherein said agonist activates FABP which reduces free radical levels in said individual, thereby improving liver function as measured by at least one liver function test.

Preferably, the disease is cholestasis and the PPAR agonist is clofibrate.

In another aspect of the invention, there is provided a method of treating an individual having a disease characterized by free radical damage an effective amount of a PPAR agonist.

As will be appreciated by one of skill in the art, the exact dosage that constitutes an effective amount will depend on several factors for a given patient, including but by no means limited to the age, weight and condition of the patient as well as the disease and the PPAR agonist used in the treatment. As used herein, “PPAR agonist” refers to PPARα agonists, PPARγ agonists and PPARΔ agonists.

As discussed above, it was previously shown by the inventors that FABP may act as an antioxidant in single cell studies. However, this did not guarantee that FABP could be used as a treatment in animal disease but rather only that this warranted further research. Furthermore, the cell line that was developed for these initial experiments had more of the nascent antioxidants, meaning that the increase in L-FABP may have been circumstantial and not the result of a direct “cause and effect” relationship. However, as discussed herein, it has since been shown that L-FABP does work in disease states, for example, cholestasis.

Bile duct ligation is a typical model of biliary disease in animals, which features proliferation of bile duct epithelial cells, hepatocellular necrosis and apoptosis, stellate cell activation and eventually the formation of liver fibrosis and cirrhosis (Kountouras et al., 1984; Scobie and Summerskill, 1965). Bile duct ligation has been associated with hepatic mitochondrial dysfunction that includes oxidative damage to mitochondrial proteins and lipids and cytotoxicity of bile components such as the lipophilic bile acids (Hino et al., 2001; Krähenbühl et al., 1994a; Krähenbühl et al., 1994b). The detergent action and cytotoxicity of bile salts is partly responsible for the plasma membrane damage seen in bile duct ligated models which leads to further oxidative stress (Sokol et al., 1995). Extensive oxidative damage results from the release of reactive oxygen species (free radicals) that most likely result from a lack of adequate reactive oxygen species scavengers. For example, hepatic glutathione in bile duct ligated animals shows a continuous decrease in reduced glutathione (GSH) and an increase in oxidized glutathione (GSSG) levels (Baron and Muriel, 1999; Neuschwander-Tetri et al., 1996; Purucker et al., 1998). Hepatic ubiquinones (lipophilic membrane associated antioxidants) also were reported to be decreased in bile duct ligated animals (Krahenbuhl et al., 1995), as were the enzyme activities of cytosolic water soluble antioxidants such as superoxide dismutase, catalase and glutathione peroxidase (Orellana et al., 2000). While the bile duct ligated model is known to be associated with reduced absorption of vitamin E, exogenous antioxidants such as vitamin E or trolox, vitamin C and silymarin do not seem to prevent the damage induced by bile duct ligation (Baron and Muriel, 1999; Muriel and Moreno, 2004). Bile duct ligation also is associated with decreased uptake of long chain fatty acids (De Vriese et al., 2001) and decreased microsomal and peroxisomal fatty acid metabolism which may partly be due to reduced amounts of peroxisomes (Orellana et al., 1997a; b). Since processing of intracellular long-chain fatty acids is disrupted, it is likely that liver fatty acid binding protein is involved in this disease state. Our results are consistent with this notion. Liver fatty acid binding protein levels were observed to be statistically lower by day 2 of bile duct ligation and declined with time following bile duct ligation. The decrease in protein level may, however, be due to the reduced number of hepatocytes rather than a decline in liver fatty acid binding protein level within the existing hepatocytes. It is known that liver weight increases with the proliferation of biliary epithelial cells and other cell and tissue types, but the volume proportion of hepatocytes is reduced (Gall and Bhathal, 1990). The reduced liver fatty acid binding protein levels (65% to 90%) in bile duct ligated rats compared with control rats one to four weeks after surgery greatly exceeded the 10% to 30% loss of hepatocyte fraction in bile duct ligated rats at the same period after surgery (Gall and Bhathal, 1990; Krahenbuhl et al., 1996; Yamauchi et al., 1976). Thus, we conclude that the dramatic reduction in liver fatty acid binding protein level in bile duct ligated animals was related to reduced protein level within the existing hepatocytes. This reduction is due to reduced liver fatty acid binding protein transcription.

Since liver fatty acid binding protein forms a large portion of the intracellular protein pool, together with its methionine and cysteine residues and great binding capacity, liver fatty acid binding protein is likely an important intracellular antioxidant. Work within our group has demonstrated that the protein likely protected hepatocytes against oxidative stress induced by H₂O₂ and hypoxia-reoxygenation in a liver fatty acid binding protein stably transfected Chang liver cell line (Wang et al., 2005). In that report we observed that cells expressing a higher level of liver fatty acid binding protein were associated with significantly reduced levels of reactive oxygen species within the cells, suggesting that the reduced reactive oxygen species levels was likely due to the increased protein level. The current study also indicated that liver fatty acid binding protein may act as an antioxidant in liver injury induced by bile duct ligation. The significant decease in protein level occurred 2 days following bile duct ligation, which preceded the decrease in (SH that is known to occur 5 days following bile duct ligation (Purucker et al., 1998). Although thiobarbituric acid-reactive substances activity started to increase at day 1 following bile duct ligation, the increase was more pronounced on day 3 and showed statistical significance by day 14 following bile duct ligation. It is likely that the antioxidant capacity of hepatocytes is able to deal with the increased levels of reactive oxygen species within the first few days of bile duct ligation. However, long term the antioxidant complement is unable to maintain a low reactive oxygen species level. Interestingly, restoration of liver fatty acid binding protein expression in the liver by clofibrate (a peroxisome proliferator) reduced the reactive oxygen species levels which was accompanied with reduced serum total bilirubin, alanine aminotransferase and ammonia. Although ammonia could increase in plasma from intestinal bacterial proliferation following bile duct ligation, the difference between bile duct ligation and bile duct ligation plus clofibrate data suggests that the ammonia likely reflects liver cell damage rather than intestinal origin. Ciprofibrate treatment has been reported to significantly reduce the activities of hepatic antioxidant enzymes NAD(P)H quinine oxidoreductase, glutathione S-transferase, glutathione peroxidase and superoxide dismutase, but increase total cellular catalase activity in rats (Dhaunsi et al., 1994; Mesia-Vela et al., 2004). Several observations indicate that clofibrate treatment contributes to a heightened defense against oxidative stress in liver (Mesia-Vela et al., 2004; Nicholls-Grzemski et al., 2000; Rajaraman et al., 2006). Nicholls-Grzemski et al. suggested that a putative antioxidant protein may contribute to this protection against liver toxicity (Nicholls-Grzemski et al., 2000). The antioxidant factor was not glutathione, but was inactivated by proteases and heat treatment. Thus, collectively with our previous report (Wang et al., 2005), our current results lead us to suggest that liver fatty acid binding protein P likely acts as an antioxidant protein. Its antioxidation function may be due to the binding of peroxidized fatty acids, bile salts and/or scavenging of reactive oxygen species through its cysteine and methionine groups. Liver fatty acid binding protein compensates for the decreased activity of the nascent antioxidant systems. Although clofibrate treatment improved liver function by upregulating liver fatty acid binding protein expression, the resulting morphological changes of liver associated with bile duct ligation were not found to be improved over the treatment period in this study.

The direct antioxidant function of liver fatty acid binding protein is likely due to its amino acid composition. The protein possess one cysteine residue at position 69 and seven methionine residues in its 127 amino acid sequence. Cysteine may be involved in the binding of other hydrophobic ligands or serve as an antioxidant participating in S-thiolation/dethiolation (Sato et al., 1996), while methionine residues have nucleophilic sulfure atoms and are regarded as a cellular scavenger of activated xenobiotics such as carcinogens (Bassuk et al., 1987). Cellular oxidative stress may be suppressed by oxidation of methionine in liver fatty acid binding protein to sulfoxides, that are then reduced back by the protein methionine sulfoxide reductase (Levine et al., 1999). This cyclic oxidation-reduction of protein methionine residues may serve an important antioxidant function (Levine et al., 1996). The net effect of these catalytic reactions is the conversion of reactive oxygen species to innocuous products, driving NADPH oxidation reactions (Stadtman, 2004). Liver also has a high expression level of methionine sulfoxide reductase (Moskovitz et al., 1996) providing for the catalytic methionine redox homeostasis. Moreover, liver fatty acid binding protein binds metabolic oxidative products such as oxidized fatty acids (Ek-Von Mentzer et al., 2001; Raza et al., 1989) and inactivates these reactive molecules. For these reasons we suggest that liver fatty acid binding protein serves as an endogenous cellular protectant, participating as a scavenger of highly reactive products resulting from metabolic reactions and/or binding of products that induce cellular oxidative damage on the surface of membranes or in the cytosol. Further studies are required to delineate the mechanism of its antioxidant function. In conclusion, our data lead us to suggest that decreased expression of liver fatty acid binding protein contributes to hepatic oxidative stress and that the protein plays an important role in the pathogenesis of biliary disease.

It is known that L-FABP is an important carrier for a variety of substances. It carries LCFA to mitochondrial and peroxisomes (Kaikaus et al., 1993, J Biol Chem 268: 26866-26871; Reubsaet et al., 1990, FEBS Lett 267: 229-230). Increase in L-FABP abundance by clofibrate is related to increased fatty acid uptake (Burczynski et al., 1999, Can J Phys Pharm 77: 896-901) and fatty acid β-oxidation (Kaikaus et al., 1993). It was documented that short-term cholestasis (BDL) in rats impaired mitochondrial ketogenesis, then disturbed hepatic fatty acid metabolism (Lang et al. 2002, J Hepatol 37: 564-571). L-FABP is an important determinant of hepatic lipid composition and turnover, and contributes to cytosolic fatty acid binding capacity, hepatic fatty acid oxidation and ketogenesis (Martin et al., 2003, J Biol Chem 278: 21429-21438: Erol et al., 2004, FASEB J 18: 347-349). Therefore, L-FABP may provide protection to mitochondria against oxidative damage by lipid accumulation. Reduction in L-FABP level will likely facilitate the mitochondrial production and accumulation of free radicals. Those free radicals could add to the peroxide pool of liver tissue. With a continued increase fatty acid intake, the liver would increase its load of free radicals (Sokol et al., 1991, J Lipid Res 32: 1349-1357). This information was consistent to our observation that BDL rats fed a high fat diet had higher mortality rates than control animals receiving a normal diet. Moreover, high mortality was associated with a large reduction of L-FABP in the liver.

The BDL model of liver disease is probably one of the best models of biliary disease. Using this model we conclude that the decreased expression of L-FABP contributes to hepatic oxidative stress and plays an important role in the pathogenesis of biliary disease. We speculate that L-FABP could be an important factor for survival in patients with biliary obstruction and high fat diet.

The invention will now be explained by way of examples, however, the invention is not limited to the examples and it is to be understood that the examples are for illustrative purposes.

### Materials and Methods

Materials: Trizol LS Reagent was purchased from GIBCO/BRL (Burlington, ON). All other chemicals were purchased from Sigma-Aldrich Canada LTD (Oakville, ON). Rat L-FABP antibody was raised in our lab (Wang et al., 2004). Rabbit anti-rat IgG and the enhanced chemiluminescence Western blot kit were purchased from Amersham-Pharmacia Biotech Inc. (Baie d'Urfe, Quebec). Advantage RT-for-PCR Kit, Advantage cDNA PCR Kit and Polymerase Mix were purchased from Clontech Laboratories Inc. (Palo Alto, Calif.). Male Sprague-Dawley rats (250-350 g) were obtained from the Animal Breeding Facility of the University of Manitoba. All rats were maintained under 12-hour light/dark cycles with food and water ad libitim. In conducting the research described in this report, all animals received humane care in compliance with institutional guidelines, which are in accordance with criteria set by the Canadian Council on Animal Care.
Bile-duct ligation animal model time study: Adult male Sprague-Dawley rats were divided into two groups (n=4 for each time point in each group); group 1 included sham operated animals and group 2 included common bile duct ligated animals as described by (Shen et al., 2005). Blood by cardiac puncture at different time points was obtained just prior to animals being sacrificed while the animals were anesthetized with ether. Livers were removed and immediately frozen in liquid nitrogen. Liver and serum samples were stored at −70° C. until required.
Bile-duct ligation animal model with clofibrate treatment: Adult male Sprague-Dawley rats were divided into three groups (n=5 for each group; total time for each group was 12 days); group 1—sham operated; group 2—common bile duct ligation as described by (Shen et al., 2005); group 3—common bile duct ligation and clofibrate treatment (50 mg/100 g body weight/day) for 5 days after the bile duct had been ligated for 7 days (total time was 12 days). Clofibrate was suspended in glycerol and administrated (1.0 ml) by gastric gavage. Control animals were administered the same volume of glycerol (without drug) by gastric gavage. Blood was sampled by cardiac puncture at different time points just prior to animals being sacrificed while the animals were anesthetized with ether. Livers were removed and immediately frozen in liquid nitrogen. Liver and serum samples were stored at −70° C. until required.
Histopathologic examination: Sections of liver tissue from sham and bile duct ligated animals were fixed in 10% buffered formalin. The fixed tissues were embedded in paraffin and then sectioned and stained with Hematoxylin and Eosin for histopathological examination. Slides were reviewed for degree of bile duct proliferation and liver inflammation.
Reverse transcriptase polymerase-chain reaction (RT-PCR): Total RNA was isolated by Trizol LS reagent as described in the manufacturer's instruction and the first cDNA strand synthesis and PCR procedure were followed. The specific primers for rat liver fatty acid binding protein were designed from the rat liver fatty acid binding protein sequence (GenBank M35991) by Oligo 5.1 program on a Macintosh computer. The sense primer was 5′-GGA AAC CTC ATT GCC ACC A-3′ (SEQ ID NO: 1) (position at 22) and the antisense primer was 5′-GCC TTG TCT AAA TTC TCT TGC TGA-3′ (SEQ ID NO: 2) (position at 407). The expected size of products was 409 base pairs. Specific rat glyceraldehyde 3-phosphate dehydrogenates amplimers were obtained from Clontech (Cat. No. 5507-3) with an expected size of 986 base pairs. PCR amplification was carried out by applying 30 cycles comprising of denaturation at 94° C. for 1 minute, annealing at 56° C. (liver fatty acid binding protein) and 60° C. (glyceraldehyde S-phosphate dehydrogenates) for 30 seconds, elongation at 72° C. for 45 seconds, followed by a final elongation at 72° C. for 8 minutes using Eppendoff MasterCycler (Eppendoff, Westbury, N.Y.). PCR products were analyzed by electrophoresis in a 1.2% agarose gel.
Western blot analysis: Liver protein was isolated by homogenizing liver tissue in Phosphate Buffered Saline (containing 1% of Protease Inhibitor Cocktail, Sigma) using a Tissue Tearor homogenizer. Protein concentration was determined by the Lowry protein assay (Lowry et al., 1951) and 20 μg of protein was subjected to Western blot analysis. Specific liver fatty acid binding protein was determined by incubating membranes with the antibody against rat liver fatty acid binding protein and bands were visualized using the enhanced chemiluminescence kit as outlined by the manufacturer's instruction.
Lipid peroxidation (thiobarbituric acid-reactive substances) assay, Supernatants from homogenized livers were subjected to a modified lipid peroxidation assay (Chirico et al., 1993). Liver protein fraction (2 mg) was added to 1.5 ml micro centrifuge tube containing 25 μl ethanolic antioxidant butylated hydroxytoluene (2 g/liter). To the tube was added 750 μl 0.44 M H₃PO₄ and the volume of the mixture adjusted to 1000 μl with doubled-distilled water. The mixture was set aside for 10 min at room temperature and then 250 μl thiobarbituric acid (6 g/L ethanol solution) added. The mixture was vortexed and heated to 100° C. for 30 min. The mixture was subsequently cooled on ice for 5 min and then centrifuged at 1000 g for 10 min. To eliminate the hemolyzed and icteric interference or other possible chromogens from thiobarbituric acid malondialdehyde adducts at wavelength 532 nm, the cooled sample was subjected to a high-performance liquid chromatography. The thiobarbituric acid reaction mixture (20 μl) was injected onto an reversed-phase C-18 column (Xterra MS, 4.6×250 mm) in a Waters 2690 alliance HPLC system (Waters Corporation, USA) and eluted with 50% 50 mM KH₂PO₄-KOH at pH 7.0 and 50% methanol at a flow rate of 0.75 ml/min. Absorbance was read at 532 nm with the retention time (R_{T}) at 5.8 min. Malondialdehyde standard (1,1,3,3-tetramethoxypropane; Sigma) was used to construct the standard curve. The degree of lipid peroxidation was expressed as concentration of thiobarbituric acid-reactive substances in terms of malondialdehyde equivalents per gram of liver protein.
Serum Total Bilirubin, Alanine Aminotransferase and Ammonia assay. Hepatic function was assessed by determining serum bilirubin, alanine aminotransferase and ammonia. Total bilirubin was determined by Sigma Diagnostic kit (550-A). Serum alanine aminotransferase and ammonia was assayed by a commercially available kit (Diagnostic Chemicals Limited). All assays were performed in a Cary Win UV spectrometer at 25° C.

### Results

### Liver Injury Following Bile Duct Ligation

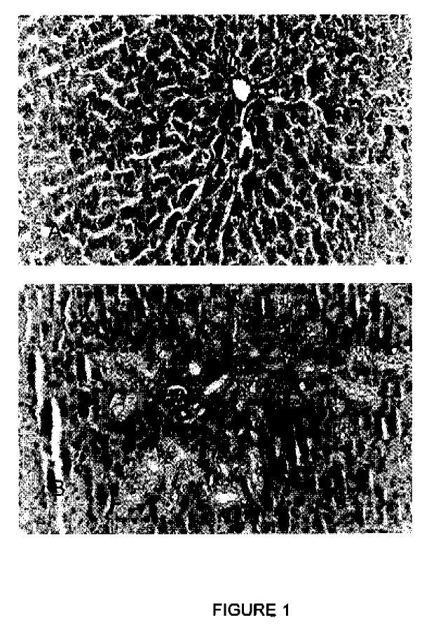

As shown in FIG. 11 bile duct proliferation and mononuclear cell infiltration were detected in the portal area of the bile duct ligated rat liver sections. Macrovascuolar cytoplasmic alterations of hepatocytes and many lysed cell areas (arrows in FIG. 1) were observed in this group. Bile duct ligation also was associated with significant proliferation of bile duct epithelial cells, inflammation and altered liver structure (circle arrow in FIG. 1).

### Liver Fatty Acid Binding Protein Expression in Bile Duct Ligated Rats

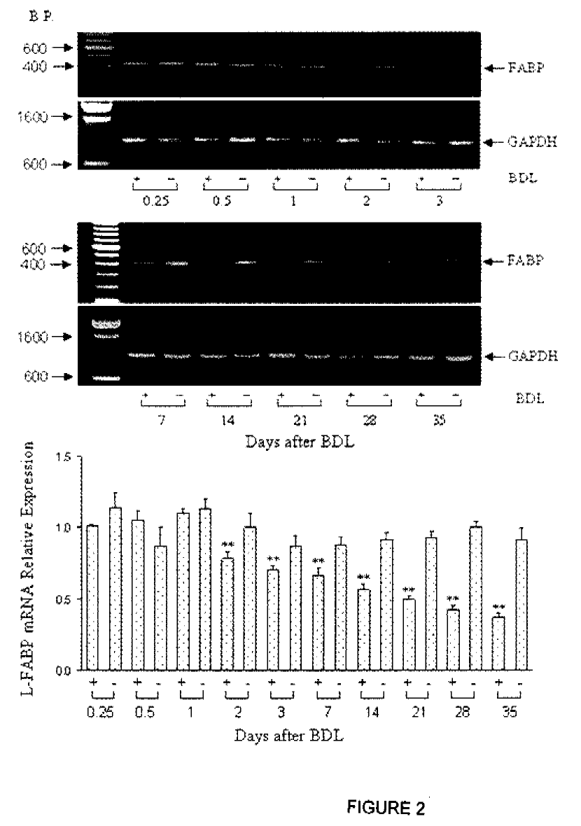

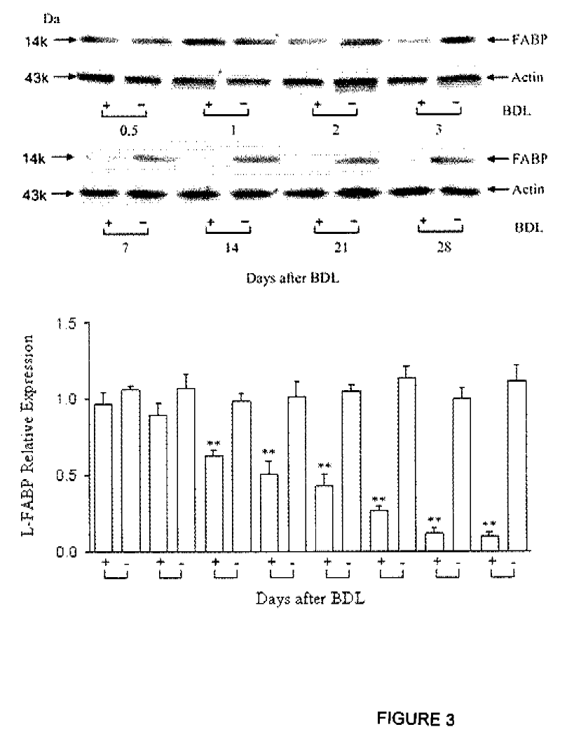

Liver fatty acid binding protein mRNA and protein level determined at different time points following bile duct ligation in rat liver tissue. Liver fatty acid binding protein mRNA expression started to decline significantly (P<0.05) after 2 days of bile duct ligation. A dramatic decrease in the liver fatty acid binding protein mRNA expression was observed after one week of bile duct ligation (FIG. 2). Reduction in liver fatty acid binding protein mRNA abundance caused a decrease in protein level. After two weeks of bile duct ligation, liver fatty acid binding protein level was almost undetectable by Western blot (FIG. 3).

### Thiobarbituric Acid-Reactive Substances, Serum Bilirubin, Alanine Aminotransferase and Ammonia Levels of Rats

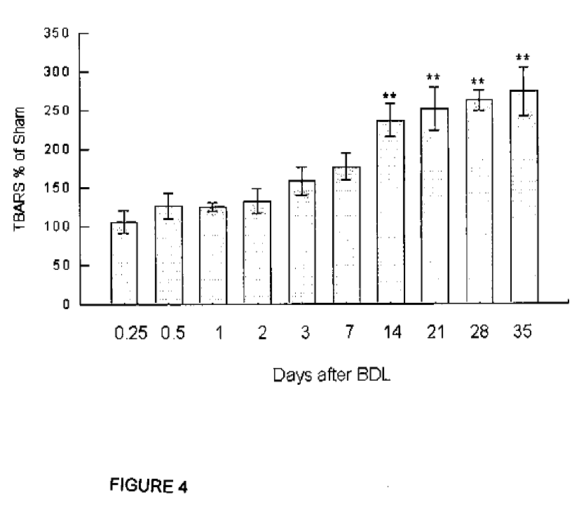

Serum bilirubin, alanine aminotransferase and ammonia levels were examined in sham and bile duct ligated rats (Table 1). Bile duct ligation was associated with a significant increase in serum bilirubin, alanine aminotransferase and ammonia (increased by 3760%, 246% and 266%, respectively) reflecting liver dysfunction. Following clofibrate treatment, serum bilirubin, alanine aminotransferase and ammonia levels were are statistically reduced. We used the thiobarbituric acid-reactive substances assay to assess lipid peroxidation levels and tissue oxidative stress. As shown in FIG. 4, the thiobarbituric acid-reactive substances increased following bile duct ligation in a time dependent manner. A significant (P<0.01) increase occurred after 14 days of bile duct ligation, indicating animals were experiencing hepatic oxidative stress and liver dysfunction. The increase in thiobarbituric acid-reactive substances production was closely associated with the decrease in liver fatty acid binding protein content.

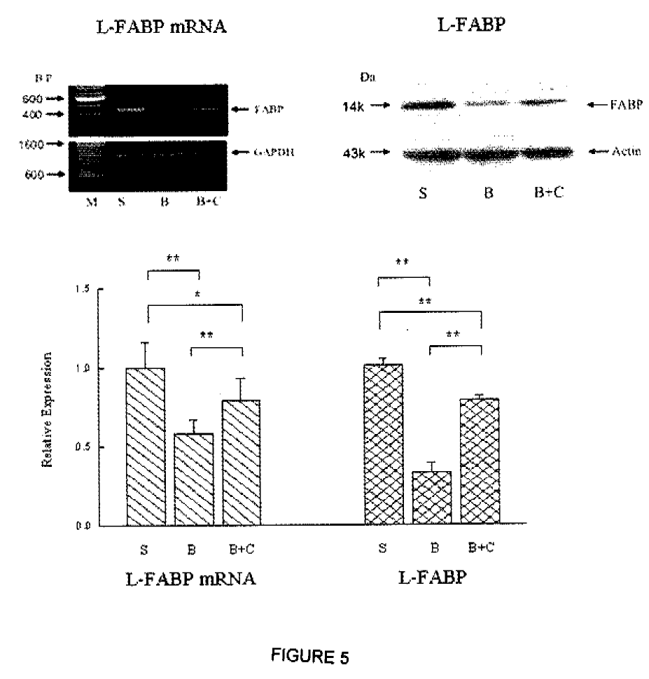

To further elucidate the association between thiobarbituric acid-reactive substances production and liver fatty acid binding protein, we examined whether the reduction in liver fatty acid binding protein may be responsible for the for some of the released liver lipid peroxides. To this end, rats were administered clofibrate to up-regulate liver fatty acid binding protein expression. Liver fatty acid binding protein expression is known to be regulated by PPAR agonists such as clofibrate (Burczynski et al., 1999). As shown in FIG. 5, after bile duct ligation liver fatty acid binding protein mRNA level was reduced to 58% as compared to the sham group while the protein level was reduced by 70% compared to the sham group (P<0.01, n=5). Five day treatment with clofibrate restored both the liver fatty acid binding protein mRNA and protein level back to 74% and 79% of the sham group, respectively. Compared to the bile duct ligated group, the bile duct ligated clofibrate treated animals had significantly elevated the mRNA and protein levels (P<0.01, n=5). The restoration of liver fatty acid binding protein level was associated with reduced lipid peroxidation products in both serum and liver (Table 1). Moreover, the up-regulation of liver fatty acid binding protein was associated with a significant decrease in serum billirubin, alanine aminotransferase and ammonia levels by 193%, 139% and 133%, respectively, as compared to bile duct ligated only animals (Table 1). These data indicate that liver fatty acid binding protein may likely be involved in the reduction of hepatic oxidative stress and improvement of hepatic function in bile duct ligated rats.

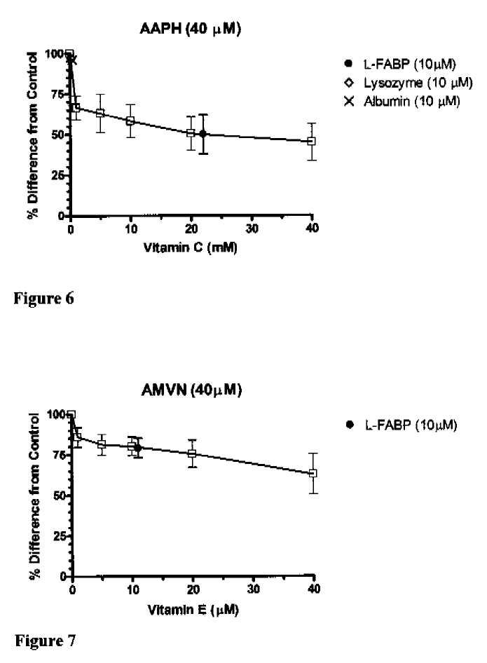

Our group has constructed and isolated recombinant L-FABP and used this protein in an in vitro model to study it's antioxidative properties. Hydrophilic free radicals were generated using 2,2′-azobis(2-amidinopropane) dihydrochloride (abbreviated AAPH) while lipophilic free radicals were generated using 2,2′-azobis(2,4-dimethylvaleronitrile (abbreviated AMVN). The extent of the L-FABP antioxidative effect was compared to albumin (MW 69000), lysozyme (MW 15000), Vitamin C, and Vitamin E. FIG. 6 shows that our recombinant protein had significantly more antioxidative effect than albumin and lysozyme when free radicals were generated using AAPH. It had as much protective effect against hydrophilic free radicals as 22 mM Vitamin C. Lysozyme, a 15 kDa protein and Albumin, a 69000 kDa protein had very little to no effect on free radical inactivation (96±1% and 97±1%, respectively, mean±SD, n=6) in this model. FIG. 7 shows similar data using the lipophilic free radical generator AMVN. Using AMVN L-FABP had significant antioxidant activity as 11 μM Vitamin E while Vitamin C had no antioxidant properties in the AMVN system.

While the preferred embodiments of the invention have been described above, it will be recognized and understood that various modifications may be made therein, and the appended claims are intended to cover all such modifications which may fall within the spirit and scope of the invention.

TABLE 1
Serum levels of bilirubin, ammonia, and TBARS and liver
TBARS level in sham, BDL, and BDL + Clofibrate rats.
Data are presented as mean ± standard error, n = 4, p < 0.05

| | Sham | BDL + Clofibrate | BDL |
|---|---|---|---|
| Total Bilirubin | 0.25 ± 0.07± | 4.87 ± 0.90 | 9.40 ± 0.51 |
| (mg/dL) | | | |
| ATL (U/L) | 17.43 ± 0.99 | 30.70 ± 3.26 | 42.67 ± 3.35 |
| Ammonia (μmol/L) | 102.6 ± 9.4 | 205.7 ± 16.6 | 273.1 ± 29.6 |
| TBARS (nmol/mg | 0.176 ± 0.041 | 0.266 ± 0.053 | 0.394 ± 0.029 |
| protein) | | | |
| TBARS (nmol/ml | 0.100 ± 0.023 | 0.212 ± 0.032± | 0.309 ± 0.039 |
| serum) | | | |

### REFERENCES

Aboutwerat, A., Pemberton, P. W., Smith, A., Burrows, P. C., McMahon, R. F., Jain, S. K., Warnes, T. W., 2003. Oxidant stress is a significant feature of primary biliary cirrhosis. Biochim Biophys Acta 1637, 142-150.
Baron, V., Muriel, P., 1999. Role of glutathione, lipid peroxidation and antioxidants on acute bile-duct obstruction in the rat. Biochim Biophys Acta 1472, 173-180.
Bassuk, J. A., Tsichlis, P. N., Sorof, S., 1987. Liver fatty acid binding protein is the mitosis-associated polypeptide target of a carcinogen in rat hepatocytes. Proc. Natl. Acad. Sci. USA 84, 7547-7551.
Burczynski, F. J., Fandrey, S., Wang, G., Pavletic, P. A., Gong, Y., 1999. Cytosolic fatty acid binding protein enhances rat hepatocyte [H-3]palmitate uptake. Canadian Journal of Physiology and Pharmacology 77, 896-901.
Burnett, D. A., Lysenko, N., Manning, J. A., Ockner, R. K., 1979. Utilization of long chain fatty acids by rat liver. Studies of the role of fatty acid binding protein. Gastroenterology 77, 241-249.
Chirico, S., Smith, C., Marchant, C., Mitchinson, M. J., Halliwell, B., 1993. Lipid peroxidation in hyperlipidaemic patients. A study of plasma using an HPLC-based thiobarbituric acid test. Free Radio Res Commun 19, 51-57.
De Vriese, S. R., Savelii, J. L., Poisson, J. P., Narce, M., Kerremans, I., Lefebvre, R., Dhooge, W. S., De Greyt, W., Christophe, A. B., 2001. Fat absorption and metabolism in bile duct ligated rats. Annals of nutrition & metabolism 45, 209-216.
Dhaunsi, G. S., Singh, I., Orak, J. K., Singh, A. K., 1994. Antioxidant enzymes in ciprofibrate-induced oxidative stress. Carcinogenesis 15, 1923-1930.
Ek-Von Mentzer, B. A., Zhang, F., Hamilton, J. A., 2001 Binding of 13-HODE and 15-HETE to phospholipid bilayers, albumin, and intracellular fatty acid binding proteins. J Biol Chem 276, 15575-15580.
Gall, J. A., Bhathal, P. S., 1990. A quantitative analysis of the liver following ligation of the common bile duct. Liver 10, 116-125.
Glatz, J. F., van der Vusse, G. J., 1996. Cellular fatty acid-binding proteins: their function and physiological significance. Prog Lipid Res 35, 243-282.
Hino, A., Morita, M., Une, M., Fujimura, K., Kuramoto, T., 2001. Effects of deoxycholic acid and its epimers on lipid peroxidation in isolated rat hepatocytes. J Biochem (Tokyo) 129, 683-689.
Hung, D. Y., Siebert, G. A., Chang, P., Burczynski, F. J., Roberts, M. S., 2005. Reduced hepatic extraction of palmitate in steatosis correlated to lower level of liver fatty acid binding protein. Am J Physiol Gastrointest Liver Physiol 288, G93-100.
Kaikaus, R. M., Sui, Z. Lysenko, N., Wu, N.Y., Ortiz de Montellano, P. R., Ockner, R. K., Bass, N. M., 1993. Regulation of pathways of extramitochondrial fatty acid oxidation and liver fatty acid-binding protein by long-chain monocarboxylic fatty acids in hepatocytes. Journal of Biological Chemistry 268, 26866-26871.
Khan, S. H., Sorof, S., 1990. Preferential binding of growth inhibitory prostaglandins by the target protein of a carcinogen. Proc. Natl. Acad. Sci. USA 87, 9401-9405.
Kountouras, J., Billing, B. H., Scheuer, P. J., 1984. Prolonged bile duct obstruction: a new experimental model for cirrhosis in the rat. Br J Exp Pathol 65, 305-311.
Krahenbuhl, L., Talos, C., Reichen, J., Krahenbuhl, S., 1996. Progressive decrease in tissue glycogen content in rats with long-term cholestasis. Hepatology 24, 902-907.
Krähenbühl, S., Tabos, C., Fischer, S., Reichen, J., 1994a. Toxicity of bile acids on the electron transport chain of isolated rat liver mitochondria. Hepatology 19, 471479.
Krahenbuhl, S., Talos, C., Lauterburg, B. H., Reichen, J., 1995. Reduced antioxidative capacity in liver mitochondria from bile duct ligated rats. Hepatology 22, 607-612.
Krähenbühl, S., Talos, C., Reichen, J., 1994b. Mechanisms of impaired hepatic fatty acid metabolism in rats with long-term bile duct ligation. Hepatology 19, 1272-1281.
Levine, R. L., Berlett, B.$., Moskovitz, J., Mosoni, L., Stadtman, E. R., 1999. Methionine residues may protect proteins from critical oxidative damage. Mech Ageing Dev 107, 323-332.
Levine, R. L., Mosoni, L., Berleft, B. S., Stadtman, E. R., 1996. Methionine residues as endogenous antioxidants in proteins. Proc Natl Acad Sci USA 93, 15036-15040.
Ljubuncic, P., Tanne, Z., Bomzon, A., 2000. Evidence of a systemic phenomenon for oxidative stress in cholestatic liver disease. Gut 47, 710-716.
Lowry, O. H., Rosebrough, N. J., Farr, A. L., Randall, R. J., 1951. Protein measurement with folin phenol reagent. Journal of Biological Chemistry 193, 265-271.
Luebker, D. J., Hansen, K. J., Bass, N. M., Butenhoff, J. L., Seacat, A. M., 2002. Interactions of fluorochemicals with rat liver fatty acid-binding protein. Toxicology 176, 175-185.
Mesia-Vela, S., Sanchez, R. I., Reuhl, K. R., Conney, A. H., Kauffman, F. C., 2004. Dietary clofibrate inhibits induction of hepatic antioxidant enzymes by chronic estradiol in female ACI rats. Toxicology 200, 103-111.
Moskovitz, J., Jenkins, N. A., Gilbert, D. J., Copeland, N. G., Jursky, F., Weissbach, H., Brot, N., 1996. Chromosomal localization of the mammalian peptide-methionine sulfoxide reductase gene and its differential expression in various tissues. Proc Natl Acad Sci USA 93, 3205-3208.
Muriel, P., Moreno, M. G., 2004. Effects of silymarin and vitamins E and C on liver damage induced by prolonged biliary obstruction in the rat. Basic Clin Pharmacol Toxicol 94, 99-104.
Neuschwander-Tetri, B. A., Nicholson, C., Wells, L. D., Tracy, T. F., Jr., 1996. Cholestatic liver injury down-regulates hepatic glutathione synthesis. J Surg Res 63, 447451.
Nicholls-Grzemski, F. A., Calder, I. C., Priestly, B. G., Burcham, P. C., 2000. Clofibrate-induced in vitro hepatoprotection against acetaminophen is not due to altered glutathione homeostasis. Toxicol Sci 56, 220-228.
Orellana, M., Avalos, N., Abarca, M., Valdes, E., 1997a. Microsomal and peroxisomal fatty acid oxidation in bile duct ligated rats: a comparative study between liver and kidney. Gen Pharmacol 28, 525-529.
Orellana, M., Avalos, N., Abarca, M., Valdes, E., 1997b. Microsomal and peroxisomal fatty acid oxidation in liver of rats with bile duct ligation and two-thirds hepatectomy. Comparative biochemistry and physiology 116, 33-37.
Orellana, M., Rodrigo, R., Thielemann, L., Guajardo, V., 2000. Bile duct ligation and oxidative stress in the rat: effects in liver and kidney. Comp Biochem Physiol C Toxicol Pharmacol 126, 105-111.
Purucker, E., Winograd, R., Roeb, E., Matern, S., 1998. Glutathione status in liver and plasma during development of biliary cirrhosis after bile duct ligation. Res Exp Med (Berl) 198, 167-174.
Rajaraman, G., Wang, G. Q., Yan, J., Jiang, P., Gong, Y., Burczynski, F. J., 2006. Role of cytosolic liver fatty acid binding protein in hepatocellular oxidative stress: effect of dexamethasone and clofibrate treatment. Mol Cell Biochem. August 19 [Epub ahead of print].
Raza, H., Pongubala, J. R., Soroft S., 1989. Specific high affinity binding of lipoxygenase metabolites of arachidonic acid by liver fatty acid binding protein. Biochem Biophys Res Commun 161, 448-455.
Sato, T., Baba, K., Takahashi, Y., Uchiumi, T., Odani, S., 1996. Rat liver fatty acid-binding protein: identification of a molecular species having a mixed disulfide with cysteine at cysteine-69 and enhanced protease susceptibility. J Biochem 120, 908-914.
Scobie, B. A., Summerskill, W. H., 1965. Hepatic Cirrhosis Secondary to Obstruction of the Biliary System. Am J Dig Dis 10, 135-146.
Shen, H., Fan, Y., Yang, X., Burczynski, F. J., Li, P., Gong, Y., 2005. Increased expression of cystic fibrosis transmembrane conductance regulator in rat liver after common bile duct ligation. J Cell Physiol 203, 599-603.
Sokol, R. J., Winklhofer-Roob, B. M., Devereaux, M. W., McKim, J. M., Jr., 1995. Generation of hydroperoxides in isolated rat hepatocytes and hepatic mitochondria exposed to hydrophobic bile acids. Gastroenterology 109, 1249-1256.
Stadtman, E. R., 2004. Cyclic oxidation and reduction of methionine residues of proteins in antioxidant defense and cellular regulation. Arch Biochem Biophys 423, 2-5.
Storch, J., Thumser, A. E. A., 2000. The fatty acid transport function of fatty acid-binding proteins. Biochimica et Biophysica Acta 1486, 2844.
Thomas, J. A., Poland, B., Honzatko, R., 1995. Protein sulfhydryls and their role in the antioxidant function of protein S-thiolation. Arch Biochem Biophys 319, 1-9.
Thompson, J., Reese-Wagoner, A., Banaszak, L., 1999. Liver fatty acid binding protein: species variation and the accommodation of different ligands. Biochim Biophys Acta 1441, 117-130.
Wang, G., Chen, Q. M., Minuk, G. Y., Gong, Y., Burczynski, F. J., 2004. Enhanced expression of cytosolic fatty acid binding protein and fatty acid uptake during liver regeneration in rats. Mol Cell Biochem 262, 4149.
Wang, G., Gong, Y., Anderson, J., Sun, D., Minuk, G., Roberts, M. S., Burczynski, F. J., 2005. Antioxidative Function of L-FABP in L-FABP Stable Transfected Chang Liver Cells. Hepatology 42, 871-879.
Yamauchi, H., Koyama, K., Otowa, T., Ouchi, K., Anezaki, T., Sato, T., 1976. Morphometric studies on the rat liver in biliary obstruction. Tohoku J Exp Med 119, 9-25.
Zimmerman, A. W., Veerkamp, J. H., 2002. New insights into the structure and function of fatty acid-binding proteins. Cell Mol Life Sci 59, 1096-1116.

1. A method of treating an individual having a disease characterized by free radical damage comprising administering to an individual in need of such treatment an effective amount of a PPAR agonist.

2. The method according to claim 1 wherein the disease characterized by free radical damage is selected from the group consisting of: cholestasis, cirrhosis, nonalcholic liver disease, fatty liver, and drug-induced free radical damage.

3. The method according to claim 1 wherein the PPAR agonist is selected from the group consisting of: linoelic acid, arachidonic acid, clofbrate, gemfibrozil, fenofibrate, ciprofibrate, bezafibrate, lovastatin, pravastatin, simvastatin, mevastatin, fluvastatin, rosiglitazone, troglitazone, indomethacin, fenoprofen, and ibuprofen.

4. The method according to claim 1 wherein the PPAR agonist is clofbrate.

5. The method according to claim 4 wherein the disease characterized by free radical damage is cholestasis.


SEQUENCE LISTING (2 sequences)

SEQ ID NO: 1 - DNA, 19 bp, artificial
  PCR primer for cloning of rat liver fatty acid binding protein
ggaaacctcattgccacca

SEQ ID NO: 2 - DNA, 24 bp, artificial
  PCR primer for cloning of rat liver fatty acid binding protein
gccttgtctaaattctcttgctga